(19) United States
(12) Patent Application Publication
(10) Pub. No.: US 2022/0339199 A1
(43) Pub. Date: Oct. 27, 2022

(54) PHARMACEUTICAL COMPOSITION FOR PREVENTING OR TREATING RETINAL DEGENERATIVE DISEASE, COMPRISING HUMAN NEURAL CREST-DERIVED NASAL INFERIOR TURBINATE STEM CELLS AS ACTIVE INGREDIENT

(71) Applicants: THE CATHOLIC UNIVERSITY OF KOREA INDUSTRY-ACADEMIC COOPERATION FOUNDATION, Seoul (KR); DIOSPHARMA CO., LTD., Chuncheon-si (KR)

(72) Inventors: Sung Won Kim, Seoul (KR); In Beom KIM, Seongnam-si (KR); Yong Soo PARK, Seoul (KR); Sang Jae SOUK, Incheon (KR)

(73) Assignees: THE CATHOLIC UNIVERSITY OF KOREA INDUSTRY-ACADEMIC COOPERATION FOUNDATION, Seoul (KR); DIOSPHARMA CO., LTD., Chuncheon-si (KR)

(21) Appl. No.: 17/659,348

(22) Filed: Apr. 15, 2022

(30) Foreign Application Priority Data
Apr. 16, 2021 (KR) 10-2021-0049821

(51) Int. Cl. A61K 35/30; A61K 9/00; A61P 27/10; A61P 25/28; A61K 38/17

(52) U.S. Cl. CPC A61K 35/30; A61K 9/0019; A61P 27/10; A61P 25/28; A61K 38/177

(57) ABSTRACT

Provided is a method for preventing or treating a retinal degenerative disease, comprising administering to a subject in need thereof human neural crest-derived nasal inferior turbinate stem cells as an active ingredient, in which the stem cells are differentiated into rod photoreceptor cells among photoreceptor cells expressing rhodopsin and prevent or treat a retinal degenerative disease including age-related macular degeneration.

### CROSS-REFERENCE TO RELATED APPLICATION

This application claims priority to and the benefit of Korean Patent Application No. 10-2021-0049821, filed on Apr. 16, 2021, the disclosure of which is incorporated herein by reference in its entirety.

### BACKGROUND

### 1. Field of the Invention

The present invention relates to a pharmaceutical composition for preventing or treating a retinal degenerative disease, comprising human neural crest-derived nasal inferior turbinate stem cells as an active ingredient.

### 2. Discussion of Related Art

The retina is a transparent structure that is sensitive to light at the back of the eyes, and the cornea and crystalline lens allow light to be focused on the retina. The central region of the retina, called the macula, includes a high density of photoreceptor cells (light sensing) that sense color. Photoreceptor cells of the retina are important optic nerve cells that receive light stimuli to convert them into electrical signals. Photoreceptor cells include rod photoreceptor cells and cone photoreceptor cells. Rhodopsin expressed in the segment of rod photoreceptor cells consists of opsin and 11-cis-retinal. After passing through the interphotoreceptor matrix, 11-cis retinal is transported to photoreceptor cells and binds to an opsin protein to form a visual pigment called rhodopsin. Since rod photoreceptor cells, which are photoreceptor cells containing rhodopsin, can capture even the smallest amount of light, the rod photoreceptor cells are important for visual acuity in the dark. Opsin expressed by cone cells produces the clearest visual images and senses central vision and color vision. The peripheral parts of the retina surrounding the macula contain rod photoreceptor cells, and respond to weak light but are not sensitive to color.

A retinal degenerative disease is a generic term for all diseases that cause pathological changes in the retina, which is a nerve tissue of the eyeball, and the macula, which plays the most important function in the retina, due to genetic abnormalities, aging, inflammation, vascular disease, and the like, resulting in blindness.

Senile macular degeneration is a disease in which changes such as drusen deposition, retinal pigment epithelial atrophy, choroidal neovascularization, and oxidative stress accumulation occur in the macula as one gets older, resulting in loss of vision, and is one of the most common causes of loss of central vision in adults over the age of 60 in developed countries including Korea. However, retinal cells, like other cells in the nervous system, are known to have poor division and regeneration ability, and when the retinal cells are lost, there is no effective method capable of regenerating or replacing the damaged cells, so there has been no fundamental preventive or curative method to date.

Meanwhile, mesenchymal stem cells may be obtained from bone marrow, sucked adipose tissue, cord blood, umbilical cord, and the like which are adult tissues, and have a fibroblast form. Since the cells can proliferate without limitation in a test tube, and can differentiate into various types of important cell series such as fat, osteocytes, chondrocytes, cardiac muscle cells, and nerve cells unlike blood stem cells, many studies have been conducted in the tissue engineering and regenerative medicine fields. Nasal inferior turbinate tissue is an independent small bone showing a shell-like shape on the lower lateral side of the nasal cavity on both the left and right sides, and is attached to the maxilla and the palatine bone. The present inventors have recently reported that mesenchymal stem cells can be isolated from discarded human nasal inferior turbinate tissue and differentiated into chondrocytes, osteocytes, adipocytes, and nerve cells (KR10-1327076). Studies in the related art have reported a problem in that proliferative vitreoretinopathy occurs as a complication when adult mesenchymal stem cells are transplanted, and a therapeutic effect of human neural crest-derived nasal inferior turbinate stem cells against a retinal degenerative disease such as retinitis pigmentosa, is not yet known.

Blue light may cause apoptosis of retinal pigment epithelial cells and photoreceptor cells by causing oxidative stress, and papers approaching from an anatomical perspective have revealed that when animals are exposed to blue light, the thickness of the outer nuclear layer of the retina, which consists of, particularly, photoreceptor cells, is decreased.

Thus, the present inventors intended to develop a therapeutic agent and a technique, which are effective for a retinal degenerative disease, using human neural crest-derived nasal inferior turbinate stem cells as an alternative for treating retinal degeneration using a retinal degenerative animal model using blue light.

### RELATED ART DOCUMENT

### Non-Patent Documents

(Non-Patent Document 1) J Korean Ophthalmic Opt Soc. 21(1):69-76, March 2016

### SUMMARY OF THE INVENTION

As a result of intensive studies to solve the aforementioned problem, the present inventors confirmed that when human neural crest-derived nasal inferior turbinate stem cells were injected into the subretinal space of mice, the injected cells were immunostained with rhodopsin constituting normal retinal rod photoreceptor cells, and thus were differentiated into rod photoreceptor cells among photoreceptor cells, thereby completing the present invention based on this.

Thus, an object of the present invention is to provide a pharmaceutical composition for preventing or treating a retinal degenerative disease, comprising human neural crest-derived nasal inferior turbinate stem cells as an active ingredient.

Further, another object of the present invention is to provide a stem cell therapeutic agent for treating a retinal degenerative disease, comprising human neural crest-derived nasal inferior turbinate stem cells as an active ingredient.

In addition, still another object of the present invention is to provide a quasi-drug composition for treating a retinal degenerative disease, comprising human neural crest-derived nasal inferior turbinate stem cells as an active ingredient. However, technical problems to be solved by the present invention are not limited to the aforementioned problems, and other problems that are not mentioned may be clearly understood by the person skilled in the art from the following description.

To achieve the aforementioned objects of the present invention, the present invention provides a pharmaceutical composition for preventing, ameliorating, or treating a retinal degenerative disease, comprising human neural crest-derived nasal inferior turbinate stem cells as an active ingredient.

Furthermore, the present invention provides a stem cell therapeutic agent for preventing, ameliorating, or treating a retinal degenerative disease, comprising human neural crest-derived nasal inferior turbinate stem cells as an active ingredient.

Further, the present invention provides a quasi-drug composition for preventing, ameliorating, or treating a retinal degenerative disease, comprising human neural crest-derived nasal inferior turbinate stem cells as an active ingredient.

As an exemplary embodiment of the present invention, the retinal degenerative disease may be one or more selected from the group consisting of retinitis pigmentosa, vitelliform macular dystrophy, Stargardt disease, X-linked retinoschisis, cone dystrophy, age-related macular degeneration, myopic choroidal neovascularization, and Behcet's uveitis, but is not limited thereto.

As another exemplary embodiment of the present invention, the human neural crest-derived nasal inferior turbinate stem cells may be characterized by being differentiated into rod photoreceptor cells among photoreceptor cells, but are not limited thereto.

As still another exemplary embodiment of the present invention, the differentiated rod photoreceptor cells may express rhodopsin, but are not limited thereto.

As yet another exemplary embodiment of the present invention, the composition may be characterized by being subretinally or intravitreally injected, but is not limited thereto.

Moreover, the present invention provides a method for preventing, ameliorating, or treating a retinal degenerative disease, the method comprising administering human neural crest-derived nasal inferior turbinate stem cells to a subject in need thereof.

Further, the present invention provides a method for preventing, ameliorating, or treating a retinal degenerative disease, the method comprising administering a cellular therapeutic agent comprising human neural crest-derived nasal inferior turbinate stem cells as an active ingredient to a subject in need thereof.

In addition, the present invention provides a method for preventing, ameliorating, or treating a retinal degenerative disease, the method comprising administering a pharmaceutical composition comprising human neural crest-derived nasal inferior turbinate stem cells as an active ingredient to a subject in need thereof.

In addition, the present invention provides a method for preventing, ameliorating, or treating a retinal degenerative disease, the method comprising administering a quasi-drug composition comprising human neural crest-derived nasal inferior turbinate stem cells as an active ingredient to a subject in need thereof. In an exemplary embodiment of the present invention, an effective amount of the human neural crest-derived nasal inferior turbinate stem cells, a cellular therapeutic agent comprising the same, or a composition comprising the same may be administered.

Furthermore, the present invention provides a use of human neural crest-derived nasal inferior turbinate stem cells or a pharmaceutical composition comprising the same as an active ingredient for preventing, ameliorating, or treating a retinal degenerative disease.

Further, the present invention provides a use of human neural crest-derived nasal inferior turbinate stem cells for preparing a medicament for preventing, ameliorating, or treating a retinal degenerative disease. The medicament may be a cellular therapeutic agent.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1A is a result showing that based on the fact that DiI-labeled human neural crest-derived nasal inferior turbinate stem cells (hTMSCs, red) were found in a subretinal space (SRS) of the retina, rhodopsin (white) was expressed in the outer segment and inner segment (OS/IS) layers and an outer segment-like structure of photoreceptor cells linked to cell bodies of the injected human neural crest-derived nasal inferior turbinate stem cells. RPE, Retinal pigment epithelium; ONL, Outer nuclear layer; OPL, Outer plexiform layer; INL, Inner nuclear layer;

FIG. 1B is a result showing that based on the fact that DiI-labeled human neural crest-derived nasal inferior turbinate stem cells (red) were found in a subretinal space (SRS) of the retina, opsin (green) was expressed in cone photoreceptors, and human neural crest-derived nasal inferior turbinate stem cells were not expressed;

FIG. 1C is an enlarged version of FIG. 1A, showing that the rhodopsin-labeled point (white) is located in a structure linked to cell bodies of human neural crest-derived nasal inferior turbinate stem cells;

FIG. 1D is an enlarged version of FIG. 1B, showing that the opsin-labeled point (green) is not located around human neural crest-derived nasal inferior turbinate stem cells; and

FIG. 1E is a high-magnification image that combines a confocal image showing immunostaining of rhodopsin and a DIC image, showing that rhodopsin is expressed in the appendages of human neural crest-derived nasal inferior turbinate stem cells.

### DETAILED DESCRIPTION OF EXEMPLARY EMBODIMENTS

As a result of subretinally injecting human neural crest-derived nasal inferior turbinate stem cells into a retinal degenerative mouse model, the present inventors confirmed that the injected human neural crest-derived nasal inferior turbinate stem cells were differentiated into rod photoreceptor cells among photoreceptor cells expressing rhodopsin, thereby completing the present invention based on this.

Hereinafter, the present invention will be described in detail.

The present invention provides a pharmaceutical composition, a stem cell therapeutic agent, and a quasi-drug composition, for preventing or treating a retinal degenerative disease, comprising human neural crest-derived nasal inferior turbinate stem cells as an active ingredient.

As used herein, the term “stem cell” refers to, as a cell that is the basis of a cell or tissue that constitutes an individual, a cell that may be repeatedly divided to achieve self-renewal and has a multi-differentiation ability to be differentiated into cells having specific functions according to the environment. Stem cells are generated in all tissues during the fetal development process, and are found in some tissues where cells are actively replaced, such as bone marrow and epithelial tissues, even in adults. Stem cells are classified into totipontent stem cells that are formed when fertilized eggs begin to divide, pluripotent stem cells that are located in the inner cell mass of the blastocyst that is created by the continuous division of these cells, and multipotent stem cells present in mature tissues and organs. In this case, multipotent stem cells are cells that can differentiate into only cells specific for tissues and organs in which these cells are included, and are involved in not only the growth and development of each tissue and organ in the fetal period, neonatal period, and adult period, but also functions of maintaining homeostasis of a living tissue and inducing regeneration during tissue damage. Such tissue-specific multipotent cells are collectively referred to as adult stem cells.

Mesenchymal stem cells classified as adult stem cells are cells that have drawn attention as a material for regenerative medicine, may be collected from tissues such as bone marrow, cord blood, and umbilical cord blood, and have the ability to differentiate into cells constituting various human body tissues such as adipose tissue cells, osteocytes, chondrocytes, nerve cells, and cardiac muscle cells unlike blood stem cells. In the present invention, mesenchymal stem cells separated from human turbinate tissue were used.

Among adult mesenchymal stem cells, bone marrow-derived mesenchymal stem cells and adipose tissue-derived mesenchymal stem cells have disadvantages in that a surgery to obtain the mesenchymal stem cells is accompanied by severe pain and is time-consuming, an amount of obtained mesenchymal stem cells is very small, a lot of time and money is spent in the process of culturing a clinically sufficient amount, and the risk of infection and cell loss is high. In addition, cord blood-derived mesenchymal stem cells have a problem in that the mesenchymal stem cells are difficult to obtain at the required time and need to be stored for a long period of time.

In contrast, human neural crest-derived nasal inferior turbinate stem cells have advantages in that a surgery to obtain the stem cells is accompanied by very little bleeding and pain and takes less time, stem cells can be continuously secured through the recycling of mesenchymal stem cells isolated from discarded inferior turbinate tissue during inferior turbinate surgery (rhinitis surgery) most frequently performed in the otorhinolaryngology area, and the ability of stem cells to proliferate is higher than that of the bone marrow-derived and adipose tissue-derived mesenchymal stem cells.

In the present invention, the retinal degenerative disease may be one or more selected from the group consisting of retinitis pigmentosa, vitelliform macular dystrophy, Stargardt disease, X-linked retinoschisis, cone dystrophy, age-related macular degeneration, myopic choroidal neovascularization, and Behcet's uveitis, but is not limited thereto.

In the present invention, the human neural crest-derived nasal inferior turbinate stem cells may be characterized by being differentiated into rod photoreceptor cells among photoreceptor cells, but are not limited thereto.

In the present invention, the differentiated rod photoreceptor cells may express rhodopsin, but are not limited thereto.

In the present invention, the photoreceptor cells may be cone photoreceptor cells and rod photoreceptor cells. Cone photoreceptor cells are relatively abundant in the central part of the retina and are responsible for vision and color vision in bright places, and rod photoreceptor cells are abundant in the peripheral part of the retina and play a role in peripheral vision and vision in dark places. In general, retinitis pigmentosa first begins with dysfunction of rod photoreceptor cells, and as degeneration progresses, abnormalities of cone photoreceptor cells also appear.

In the present invention, retinitis pigmentosa is caused by a genetic abnormality. This causes abnormalities in the normal functioning of photoreceptors and degeneration of the retina occurs, so that visual acuity deteriorates. Retinitis pigmentosa is a hereditary disease due to a genetic abnormality, but may occur without a family history.

In the present invention, the composition may be characterized by being subretinally or intravitreally injected, but is not limited thereto.

In the present invention, the composition may include a growth factor, but is not limited thereto. The growth factor may be a vascular endothelial growth factor (VEGF), a pigment epithelium-derived factor (PEDF), a brain-derived neurotrophic factor (BDNF), and the like, but is not limited thereto.

The amount of the human neural crest-derived nasal inferior turbinate stem cells in the composition of the present invention may be appropriately adjusted depending on the symptoms of a disease, the degree of progression of symptoms, the condition of a patient, and the like, and may range from, for example, 0.0001 wt % to 99.9 wt % or 0.001 wt % to 50 wt % with respect to a total weight of the composition, but the present invention is not limited thereto. The amount ratio is a value based on the amount of dried product from which a solvent is removed.

The pharmaceutical composition or quasi-drug composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition or quasi-drug composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

The stem cells, the pharmaceutical composition, the quasi-drug composition, or the cellular therapeutic agent according to the present invention may preferably have one or more formulations selected from the group consisting of an ophthalmic solution, a collyrium, an ophthalmic ointment, an injection, and an eyewash, but is not limited thereto.

When the formulation is prepared as an ophthalmic solution, the formulation may be provided in any administration form used as an ophthalmic solution, for example, an aqueous ophthalmic solution such as an aqueous emulsion ophthalmic solution, a viscous ophthalmic solution and a dissolved ophthalmic solution; or a non-aqueous ophthalmic solution such as a non-aqueous emulsion ophthalmic solution.

When the formulation is prepared as an aqueous emulsion ophthalmic solution, various additives known in the art may be included, as long as they do not impair the object of the invention, and for example, an isotonic agent, a buffer, a stabilizer, a pH adjusting agent, a thickener, a preservative, a chelating agent, a solubilizer, a solvent, and the like may be included. The buffer may be selected from the group consisting of a phosphate buffer, a borate buffer, a citrate buffer, a tartrate buffer, an acetate buffer (for example, sodium acetate), tromethamine and an amino acid, but is not limited thereto. Preferably, a phosphate buffer may be used. The isotonic agent may be selected from the group consisting of sugars such as sorbitol, glucose erythritol and mannitol, polyhydric alcohols such as glycerin, polyethylene glycol and polypropylene glycol, and salts such as sodium chloride, but is not limited thereto. The preservative may be selected from the group consisting of benzalkonium chloride, benzethonium chloride, alkyl paraoxybenzoates such as methyl paraoxybenzoate and ethyl paraoxybenzoate, benzyl alcohol, phenethyl alcohol, sorbic acid and salts thereof, thimerosal, polyquaternium, benzododecinium bromide, an oxychloro complex and chlorobutanol, but is not limited thereto. The stabilizer may be selected from the group consisting of cyclodextrin and derivatives thereof, a water-soluble polymer such as poly(vinylpyrrolidone), and a surfactant such as polysorbate 80 (Tween 80), polysorbate 20 and tyloxapol, but is not limited thereto. The pH adjusting agent may be selected from the group consisting of hydrochloric acid, acetic acid, phosphoric acid, sulfuric acid, sodium hydroxide, potassium hydroxide, monoethanolamine, ammonia water and ammonium hydroxide, but is not limited thereto. The thickener may be selected from the group consisting of hydroxyethyl cellulose, hydroxypropyl cellulose, methyl cellulose, hydroxypropylmethyl cellulose and carboxymethyl cellulose, polyvinyl alcohol, a carbomer, povidone, a poloxamer, polycarbophil and salts thereof, but is not limited thereto. The chelating agent may be selected from the group consisting of sodium edetate, sodium citrate and condensed sodium phosphate, but is not limited thereto. The solubilizer or solvent may be selected from glycerin, DMSO, DMA, N-methylpyrrolidone, ethanol, benzyl alcohol, isopropyl alcohol, polyethylene glycol or propylene glycol with various molecular weights, and the like, but is not limited thereto. There may be some overlap between components that can be used as the solvent or the solubilizer, and since any component may be used as either the solvent or the solubilizer, when any component acts as a solvent in the preparation, it may be regarded as a solvent, and when any component does not act as a solvent, it may be regarded as a solubilizer. Alternatively, the solubilizer may be a surfactant in some variations. Surfactant combinations comprising various types of surfactants may be commercially available. For example, nonionic, anionic (that is, soap, sulfonate), cationic (that is, CTAB), zwitterionic, polymeric, amphoteric surfactants may be used. For example, available surfactants include, but are not limited to, those having an HLB of 10, 11, 12, 13, or 14 or more. Examples of the surfactant include a polyoxyethylene product of hydrogenated vegetable oil, polyethoxylated castor oil or polyethoxylated hydrogenated castor oil, polyoxyl castor oil or derivatives thereof, polyoxyethylene-sorbitan-fatty acid ester, polyoxyethylene castor oil derivatives, and the like, but are not limited thereto. According to exemplary embodiments, the ophthalmic composition of the present invention may include 0.01 to 0.1 wt % of cyclosporine, 0.5 to 7.5 wt % of trehalose, 1 to 10 wt % of a solubilizer, 0.01 to 2 wt % of a solvent, the remainder as a buffer and an isotonic agent, based on the total weight of the composition.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG; lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases. Specifically, the effective amount of the composition according to the present invention may vary depending on the severity of the disease, the patient's age, sex, and body weight, and generally, 0.001 to 150 mg of the composition and preferably, 0.01 to 100 mg of the composition, per 1 kg of the body weight, may be administered daily or every other day or may be administered once to three times a day. However, since the effective amount may be increased or decreased depending on the administration route, the severity of obesity, gender, body weight, age, and the like, the dosage is not intended to limit the scope of the present invention in any way.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “improvement” as used herein means all actions that reduce the degree of parameters related to a target disease, e.g., symptoms via administration of the composition according to the present invention.

As used herein, “quasi-drug” refers to articles which have a milder action than a drug among articles used for the purpose of diagnosing, treating, ameliorating, alleviating, treating, or preventing a human or animal disease, and for example, according to the Pharmaceutical Affairs Act, the quasi-drug is an article that is not used for pharmaceutical purposes, and includes fiber and rubber products used for the treatment and prevention of human and animal diseases, those that have little or no direct effect on the human body and are similar to those that are not instruments or machines, and bactericidal and pesticides to prevent infectious diseases, and the like.

In the present invention, the quasi-drug composition may be used by being formulated into a dosage form of an opthalmic composition, and may be used, for example, as a dosage form of one or more formulations selected from the group consisting of an ophthalmic solution, a collyrium, an ophthalmic ointment, an injection, and an eyewash, but is not limited thereto.

In the present invention, the pharmaceutical composition or quasi-drug composition may be for local administration to the eyeball, and the human neural crest-derived nasal inferior turbinate stem cells may be injected into subretinal or intravitreal space of the eyes, but are not limited thereto.

In the present invention, the human neural crest-derived nasal inferior turbinate stem cells may be injected into a patient's body in cells alone or in a state of being cultured in an incubator.

As used herein, the term “cellular therapeutic agent” refers to a drug used for the purpose of treatment, diagnosis, and prevention, using a cell or tissue prepared through isolation from a human, culture and specific manipulation (US FDA regulations), and specifically, it refers to a drug in which these cells are used for the purpose of treatment, diagnosis, and prevention through a series of actions such as multiplying and selecting living autologous, allogenic and xenogenic cells in vitro or changing the biological characteristics of cells by other methods in order to recover the functions of cells or tissues. The cellular therapeutic agent of the present invention comprises a stem cell therapeutic agent (that is, stem cellular therapeutic agents.)

The cellular therapeutic agent according to the present invention may be prepared in the form of a unit dose or by being contained in a multi-dose container by being formulated using a pharmaceutically acceptable carrier and/or excipient according to a method that can be readily carried out by a person with ordinary skill in the art to which the present invention pertains. A pharmaceutically acceptable carrier included in the cellular therapeutic agent of the present invention is a pharmaceutically acceptable carrier typically used in formulation, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, or the like, but is not limited thereto. The cellular therapeutic agent of the present invention may additionally include a lubricant, a wetting agent, an emulsifier, a suspending agent, a preservative, and the like, in addition to the aforementioned ingredients.

In addition, the composition may also be administered by any device which may allow a cellular therapeutic agent to move to a target cell.

The cellular therapeutic agent composition of the present invention may include a therapeutically effective amount of a cellular therapeutic agent for treating a disease. The “therapeutically effective amount” refers to an amount of active ingredient or pharmaceutical composition which induces a biological or medical response in a tissue system, animal or human considered by a researcher, veterinarian, physician or other clinicians, and this includes an amount that induces alleviation of the symptoms of a disease or disorder being treated.

It is obvious to those skilled in the art that the cellular therapeutic agent included in the composition of the present invention is changed depending on a desired effect. Therefore, an optimal content of the cellular therapeutic agent may be easily determined by those skilled in the art, and may be adjusted by various factors including the type of disease, the severity of disease, the content of other ingredients contained in the composition, the type of dosage form, and the age, body weight, general health status, gender and [symbol] diet of a patient, the administration time, the administration route and the excretion rate of a composition, treatment period, and simultaneously used drugs. It is important to include an amount that can obtain the maximum effect with the minimum amount without side effects in consideration of all of the above factors. For example, as a daily dose of stem cells of the present invention, 1.0×10⁴ to 1.0×10¹¹ cells/kg body weight, preferably 1.0×10⁵ to 1.0×10⁹ cells/kg body weight may be administered once or divided into several doses. However, it is to be understood that the actual dose of the active ingredient needs to be determined in consideration of various related factors such as the disease to be treated, the severity of the disease, the administration route, the body weight, age and gender of a patient, and accordingly, the dose does not limit the scope of the present invention in any way.

Hereinafter, preferred examples for helping the understanding of the present invention will be suggested. However, the following examples are provided only to more easily understand the present invention, and the contents of the present invention are not limited by the following examples.

### Example 1. Culture of Human Neural Crest-Derived Nasal Inferior Turbinate Stem Cells

With the consent of a patient before surgery, an inferior turbinate tissue was collected during inferior turbinectomy, and immediately after the inferior turbinate tissue was collected, fibroblasts were separated by washing the tissue with physiological saline containing gentamicin (Kukje Pharma. CO., LTD., Seongnam, Korea) three to five times.

In order to isolate human neural crest-derived nasal inferior turbinate stem cells from the inferior turbinate tissue collected by the above process, the collected tissue was first stored in refrigerator at 4° C. and then washed three times with an antibiotic-antifungal solution (Gibco, Gaithersberg, Md.) at room temperature. Then, the tissue was again washed twice with neutral phosphate buffered saline (PBS), and then finely cut into a size of 1 mm³ using surgical scissors.

The cut tissue was placed on a 100-mm culture dish, covered with a sterilized slide glass, adhered to the culture dish, and cultured in an incubator under an environment of 37° C. and 5% CO₂ by adding a Dulbeco's Modified Eagle's Media (DMEM) supplemented with 10% fetal bovine serum (FBS) thereto. After the tissue was cultured for 2 to 3 weeks, the slide glass was removed, cells floating in the culture solution were washed and discarded, human neural crest-derived nasal inferior turbinate stem cells attached to the bottom of the culture dish were detached from the bottom, and cells that were subcultured up to the third generation were used.

### Example 2. Confirmation of Therapeutic Effect of Human Neural Crest-Derived Nasal Inferior Turbinate Stem Cells (hTMSCs) on Retinal Degeneration

1 microliter (1×10⁶ cells/100 μl) of a DiI-labeled hTMSC suspension was injected into the subretinal space of BALB/c mice, in which retinal degeneration (RD) was induced by exposing the mice to a 2000 lx blue LED for 2 hours. 14 days after injection, eyecups were prepared, fixed in 4% paraformaldehyde and embedded for frozen sectioning. Vertical sections of the retina were immunostained for rhodopsin and opsin, known as a rod photoreceptor cell marker and a cone photoreceptor cell marker, respectively. Sections were observed using a confocal microscope (LSM 800 with Airyscan; Carl Zeiss Co. Ltd., Oberkochen, Germany) with a differential interference contrast (DIC) filter.

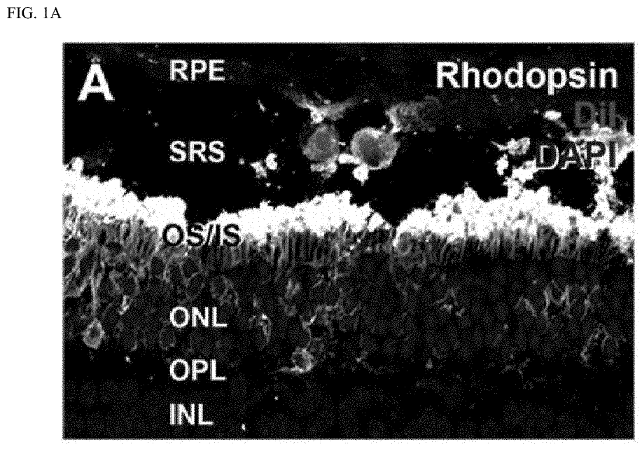

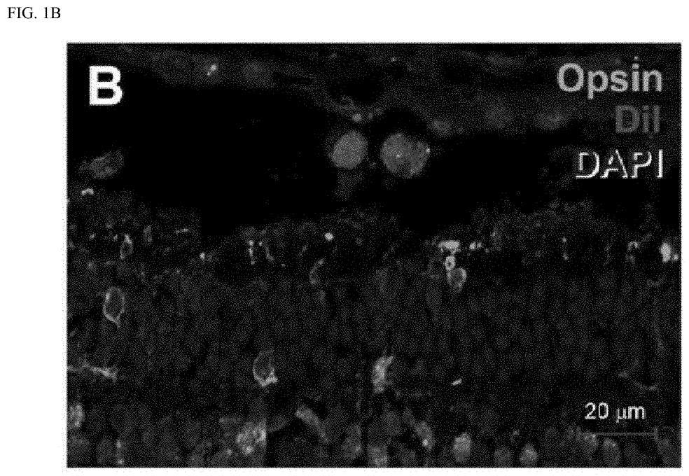

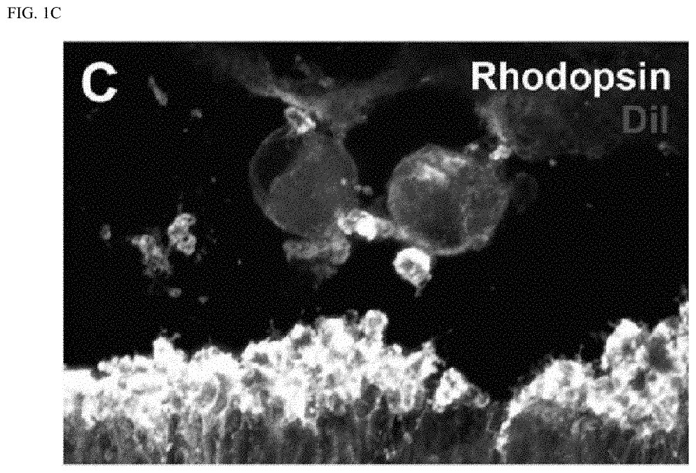

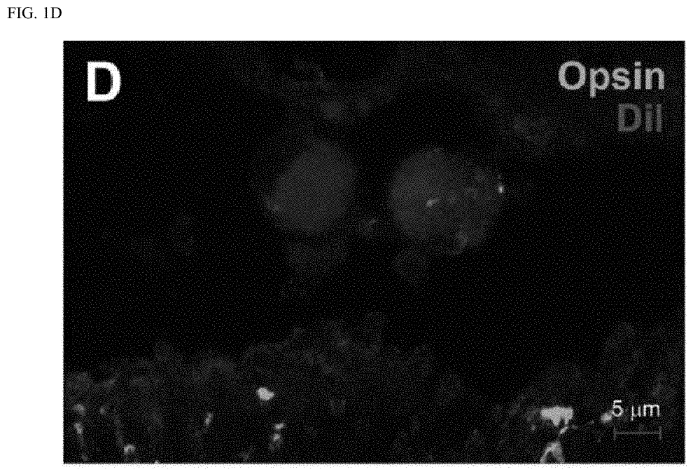

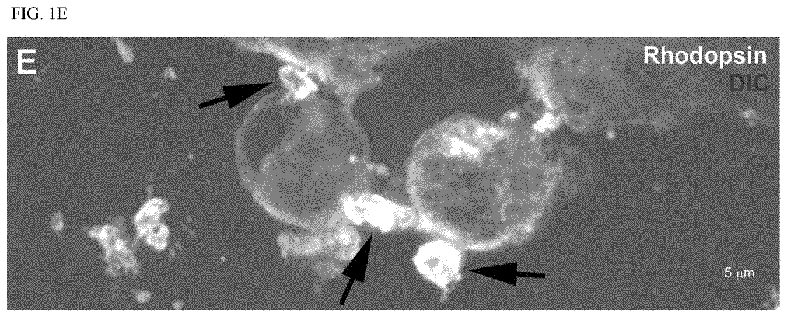

As illustrated in FIGS. 1A and 1B, it was confirmed rhodopsin (white) was mainly expressed in the outer part of photoreceptor cells, and opsin (green) was expressed in cone photoreceptor cells. Further, since mice are rod-dominant animals, it was found that most photoreceptors express rhodopsin rather than opsin.

In addition, as illustrated in FIG. 1C, rhodopsin was found to be located in a structure linked to the injected hTMSC cells in a high-magnification image.

In contrast, as illustrated in FIG. 1D, opsin was not detected around or inside the hTMSC cells.

Furthermore, a DIC image was obtained to confirm whether rhodopsin was expressed in the injected cells or particles separated from the segment layer of the retina.

As illustrated in FIG. 1E, in the image merged with the DIC image, it was confirmed that rhodopsin-labeled points (marked with black arrows) are accessory structures linked to the injected human neural crest-derived nasal inferior turbinate stem cells. This indicates that rhodopsin is expressed in the injected stem cells.

The above results indicate that human neural crest-derived nasal inferior turbinate stem cells may differentiate into rod photoreceptor cells on the degenerated retina.

From the above results, it is expected that human neural crest-derived nasal inferior turbinate stem cells will be used as a stem cell therapeutic agent for retinal degeneration.

As a result of subretinally injecting human neural crest-derived nasal inferior turbinate stem cells into a retinal degenerative mouse model, the present inventors confirmed that the injected human neural crest-derived nasal inferior turbinate stem cells were differentiated into rod photoreceptor cells among photoreceptor cells expressing rhodopsin. Therefore, it is expected that the human neural crest-derived nasal inferior turbinate stem cells of the present invention can be usefully used for the prevention or treatment of a retinal degenerative disease including age-related macular degeneration.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

What is claimed is:

1. A method for treating a retinal degenerative disease, the method comprising administering human neural crest-derived nasal inferior turbinate stem cells to a subject in need thereof.

2. The method of claim 1, wherein the retinal degenerative disease is one or more selected from the group consisting of retinitis pigmentosa, vitelliform macular dystrophy, Stargardt disease, X-linked retinoschisis, cone dystrophy, age-related macular degeneration, myopic choroidal neovascularization, and Behcet's uveitis.

3. The method of claim 1, wherein the human neural crest-derived nasal inferior turbinate stem cells are differentiated into rod photoreceptor cells among photoreceptor cells.

4. The method of claim 3, wherein the differentiated rod photoreceptor cells express rhodopsin.

5. The method of claim 1, wherein the human neural crest-derived nasal inferior turbinate stem cells are subretinally or intravitreally injected.

6. A method for treating a retinal degenerative disease, the method comprising administering a cellular therapeutic agent, wherein the cellular therapeutic agent comprises human neural crest-derived nasal inferior turbinate stem cells as an active ingredient to a subject in need thereof.

7. The method of claim 6, wherein the retinal degenerative disease is one or more selected from the group consisting of retinitis pigmentosa, vitelliform macular dystrophy, Stargardt disease, X-linked retinoschisis, cone dystrophy, age-related macular degeneration, myopic choroidal neovascularization, and Behcet's uveitis.

8. The method of claim 6, wherein the human neural crest-derived nasal inferior turbinate stem cells are differentiated into rod photoreceptor cells among photoreceptor cells.

9. The method of claim 8, wherein the differentiated rod photoreceptor cells express rhodopsin.

10. The method of claim 6, wherein the cellular therapeutic agent is subretinally or intravitreally injected.

11. A method for treating a retinal degenerative disease, the method comprising administering a quasi-drug composition, wherein the quasi-drug comprises human neural crest-derived nasal inferior turbinate stem cells as an active ingredient to a subject in need thereof.

12. The method of claim 11, wherein the retinal degenerative disease is one or more selected from the group consisting of retinitis pigmentosa, vitelliform macular dystrophy, Stargardt disease, X-linked retinoschisis, cone dystrophy, age-related macular degeneration, myopic choroidal neovascularization, and Behcet's uveitis.

13. The method of claim 11, wherein the human neural crest-derived nasal inferior turbinate stem cells are differentiated into rod photoreceptor cells among photoreceptor cells.

14. The method of claim 13, wherein the differentiated rod photoreceptor cells express rhodopsin.

15. The method of claim 11, wherein the composition is subretinally or intravitreally injected.
